(19) United States
(12) Patent Application Publication
(10) Pub. No.: US 2009/0131381 A1
(43) Pub. Date: May 21, 2009

(54) COMPOSITIONS COMPRISING HYDROQUINONE, FLUOCINOLONE ACETONIDE AND TRETINOIN FOR TREATING PHOTOAGING OF THE SKIN

(75) Inventor: Doris Maria HEXSEL, Porto Alegre (BR)

Correspondence Address: BUCHANAN, INGERSOLL & ROONEY PC, POST OFFICE BOX 1404, ALEXANDRIA, VA, 22313-1404, US

(73) Assignee: GALDERMA S.A., Cham (CH)

(21) Appl. No.: 12/272,110

(22) Filed: Nov. 17, 2008

(30) Foreign Application Priority Data
May 19, 2006 (FR) 0604504
May 21, 2007 (EP) PCT/EP2007/054897

(51) Int. Cl. A61K 31/58; A61P 17/16

(52) U.S. Cl. 514/171

(57) ABSTRACT

Dermatological/medicament compositions containing a combination of hydroquinone, fluocinolone acetonide and tretinoin are useful for treating the signs of photoaging of the skin.

### CROSS-REFERENCE TO PRIORITY/PCT APPLICATIONS

This application claims priority under 35 U.S.C. § 119 of FR 06/04504, filed May 19, 2006, and is a continuation/national phase of PCT/EP 2007/054897, filed May 21, 2007 and designating the United States (published in the French language on Nov. 29, 2007 as WO 2007/135132 A1; the title and abstract were also published in English), each hereby expressly incorporated by reference in its entirety and each assigned to the assignee hereof.

### BACKGROUND OF THE INVENTION

1. Technical Field of the Invention

The present invention relates to dermatological compositions comprising a combination of hydroquinone, fluocinolone acetonide and tretinoin, for treating the signs of photoaging of the skin.

2. Description of Background and/or Related and/or Prior Art

Human skin is constituted of two compartments, namely, a deep compartment, the dermis, and a surface compartment, the epidermis.

The dermis provides the epidermis with a solid support; it is also its nourishing factor. It is mainly constituted of fibroblasts and of an extracellular matrix that is itself mainly composed of collagen, elastin and a substance known as ground substance. Leukocytes, mastocytes or tissue macrophages are also situate therein. It also contains blood vessels and nerve fibers.

The epidermis is in contact with the external environment. It is composed of keratinocytes, melanocytes and langerhans cells. Its role consists in protecting the body against dehydration and external attack, whether this is chemical, mechanical, physical or infectious attack. The accumulation of these types of attack throughout life, coupled to the genetic influence, leads to aging of the skin.

Aging of the skin entails two distinct phenomena: intrinsic aging, which is the consequence of the passing of time, and photoaging, which is the consequence of exposure to sunlight. Intrinsic aging of the skin results in a finely wrinkled, lax, dry appearance optionally with the presence of benign tumors. Photoaging of the skin is, for its part, observed in areas usually exposed to sunlight, such as the face, the back of the hands and the forearms, and the neckline (Ann. Dermatol. Venereol., (2005): 132: 362-7).

The severity and the clinical aspect of photoaging of the skin are determined by the cumulative exposure to UV, and by an individual's capacity to respond to this exposure. Specifically, individuals of phototypes I and II present a combination of signs slightly different from those individuals of phototypes III to V, with matt skin.

Photoaging may be characterized by various cutaneous signs, such as skin roughness, actinic keratoses, solar elastosis, wrinkles (fine and deep), telangiectasias, mottled hyperpigmentation or other pigmentary disorders such as solar lentigo and freckles.

This phenomenon should be clearly distinguished from melasma. Specifically, melasma, which is also known as chloasma or a pregnancy mask, is an acquired form of hyperpigmentation that appears mainly on the face. It may result from a pregnancy, from the use of an oral contraceptive, from the menopause, or may occur spontaneously for no apparent reason. It is exacerbated by exposure to UV (“Hyperpigmentation disorders”, Goodheart, Women's Health in Primary Care, Vol. 2, No. 12/December 1999).

It consists of browning of facial skin by asymptomatic marks. The lesions are dark to brown, and the hyperpigmented macules may coalesce into clearly delimited symmetrical areas. They are found mainly on the cheeks, the forehead, the nose, the upper lip or the chin. The dark cutaneous area may disappear spontaneously after pregnancy, stopping the oral contraceptive or in the absence of exposure to sunlight.

Melasma should be distinguished from solar lentigo, freckles and post-inflammatory hyperpigmentation (“Hyperpigmentation disorders”, Goodheart, Women's Health in Primary Care, Vol. 2, No. 12/December 1999).

Thus, photoaging presents specific cutaneous signs, and should not be confused with melasma.

Among these cutaneous signs, lentigo consists of small macules that are found everywhere on the back of the hands and the arms, on the neckline or even on the face. These marks are darker than freckles, and persist in winter. They are due not only to an adaptation of the cells that manufacture a brown or red melanine, but also to a multiplication of the number of melanocytes. The color of these marks is uniform, and they appear with age on areas exposed to sunlight, such as the face and the back of the hands. This disorder is also known as solar lentigo.

The diffuse hyperpigmentation, also known as “mottled hyperpigmentation”, is reflected by a diffuse irregularity of the skin coloration.

Signs of actinic keratosis are also found on photoaged skin: these lesions, which are for the most part asymptomatic, may also be considered as precancerous lesions. They are localized thickenings of the skin, hyperkeratosic, and present squamae. If the patient attempts to pull off these squamae, light bleeding occurs.

The term “solar elastosis” means slackening of the skin, which loses its elasticity, due to the impairment and breaking of the elastin fibers synthesized by the dermal fibroblasts.

The term “telangiectasia” means a permanent dilation of the small blood vessels, capillaries, arterioles or venules, which trace fine red lines under the skin.

Individuals of phototypes I and II are more prone to lentigo and fine lines, whereas individuals of phototypes III to V are also prone to lentigo accompanied by deep wrinkles and by a nodular and coarse appearance of the skin corresponding to elastosis.

The prevention of photoaging has in recent years become a veritable public health challenge, due to the dramatic consequences that it may entail, i.e., an increase in the prevalence of skin cancers (melanomas).

However, at the present time, beyond prevention, there is a real need for the treatment of the signs of photoaging of the skin.

Only one topical agent has shown efficacy in correcting the signs of photoaging; this agent is tretinoin. It improves the appearance of photoaged skin, especially with an improvement in the roughness of the skin, fine lines and pigmentation. However, treatment with tretinoin is lengthy, about 6 months, and must be chronic, which does not facilitate the compliance with the treatment by the patients.

Other treatments exist at the present time, such as treatment with α-hydroxy acids, or β-hydroxy acids. However, the latter do not appear to be sufficiently effective, especially in the treatment of pigmentary marks.

Treatment by chemical peeling is, admittedly, much more effective, but cannot be considered as being free of risks. Specifically, a phenomenon of pigmentary rebound may be observed after a chemical peeling treatment.

Other techniques, such as dermabrasion or the use of various light sources (including CO₂ laser) are also employed, but are just as heavy and pose the same drawbacks as chemical peeling.

Consequently, real need exists for an effective, risk-free treatment of the symptoms of photoaging, in particular the hyperpigmentation marks induced by exposure to ultraviolet rays.

### SUMMARY OF THE INVENTION

Surprisingly, it has now been discovered that the combination of hydroquinone, fluocinolone acetonide and tretinoin in a pharmaceutical composition permits treating the signs of photoaging of the skin, in particular hyperpigmentation marks.

The present invention thus features formulation of fluocinolone acetonide, hydroquinone and tretinoin into medicaments useful for treating the signs of photoaging of the skin.

According to the invention, the term “signs of photoaging of the skin” means hyperpigmentation and non-pigmentation skin disorders induced by UV radiation.

Among the skin pigmentation disorders induced by UV radiation, actinic keratosis, mottled hyperpigmentation, solar lentigo (or senile lentigo), freckles, or flat pigmented seborrhoeic warts are especially representative.

The term “flat pigmented seborrhoeic wart” means a colored lesion (non-serious) in relief appearing mainly on the back and the thorax, and the surface of which resembles a cauliflower.

Among the non-pigmentation skin disorders induced by UV radiation, solar elastosis, wrinkles and fine lines, and skin roughness are especially representative.

Preferentially, the subject medicaments are useful for treating hyperpigmentation skin disorders.

Advantageously, the medicaments according to the present invention are suited for topical application, whether regime or regimen.

The medicaments according to the present invention also comprise a physiologically acceptable medium, i.e., a medium that is compatible with the skin, and may constitute a dermatological composition.

### DETAILED DESCRIPTION OF BEST MODE AND SPECIFIC/PREFERRED EMBODIMENTS OF THE INVENTION

Hydroquinone is a known depigmenting agent. It is prepared by reducing p-benzoquinone with sodium bisulfite. The chemical name of hydroquinone is 1,4-benzenediol.

Advantageously, hydroquinone is present in the subject medicaments at a concentration of from 1% to 10% by weight, advantageously from 2% to 7% to more advantageously at a concentration of 4% by weight relative to the total weight of the medicament.

Tretinoin is an all-trans-retinoic acid formed by oxidation of the aldehyde group of retinene into a carboxyl group. The chemical name of tretinoin is (all-E)-3,7-dimethyl-9-(2,6,6-trimethyl-1-cyclohexen-1-yl)-2,4,6,8-nonatetraenoic acid. It is highly reactive to light and moisture. Its action on keratinocyte differentiation and proliferation and on collagen synthesis and its depigmenting activity are advantageous in the treatment of photoaging.

In one particular embodiment, tretinoin is present in the medicaments according to the present invention at a concentration of from 0.025% to 2% by weight, advantageously from 0.025% to 1% by weight and even more advantageously at about 0.05% by weight relative to the total weight of the medicament.

Fluocinolone acetonide is a fluorinated synthetic corticosteroid useful for topical dermatological administration, and is employed as an anti-inflammatory agent. Its chemical name is (6,11,16)-6,9-difluoro-11,21-dihydroxy-16,17-[(1-methylethylidene)bis(oxy)]pregna-1,4-diene-3,20-dione. It is a light-stable, odorless white crystalline powder.

In one particular embodiment, fluocinolone acetonide is present in the medicaments according to the present invention at a concentration of from 0.005% to 0.1% by weight, advantageously from 0.005% to 0.05% by weight and even more advantageously at about 0.01% by weight relative to the total weight of the medicament.

Advantageously, the medicaments according to the present invention contains sodium metabisulfite to prevent the oxidation of the hydroquinone.

Additional constituents may be present in the medicaments according to the present invention in an amount of from 0.001% to 20% by weight relative to the total weight of the medicament.

The medicaments according to the present invention may comprise a wide variety of additional constituents, which may in particular be absorbents, abrasive agents, anti-acne agents, antifoams, antimicrobial agents, antioxidants, binders, biological additives, buffers, chelating agents, dyes, cosmetic astringents, cosmetic biocides, external analgesics, film-forming agents, fragrant components, opacifiers, plasticizers, preservatives, other depigmenting agents, emollients, skin-protecting agents, solvents, solubilizing agents, surfactants, ultraviolet radiation absorbers, sunscreens, viscosity-increasing agents (aqueous or non-aqueous), humectants, sequestrants, etc.

One skilled in this art will obviously take care to select the possible additional compounds and/or the amount thereof such that the advantageous properties of the medicaments according to the present invention are not entirely, or not substantially, diminished by the envisaged addition.

The medicaments according to the present invention may be provided in any galenical form normally employed in dermatology.

Preferentially, the compositions according to the present invention are in the form of a cream. The term “cream” means a water-based preparation for topical application. It corresponds to an emulsion, i.e., it comprises at least one lipophilic phase and at least one hydrophilic phase.

The creams may be advantageously prepared as indicated in WO 2004/037 201, via a process including the steps of:

a) mixing the hydrophilic compounds with water to form an aqueous or hydrophilic phase;

b) mixing the hydrophobic compounds to form a hydrophobic phase;

c) mixing the hydrophobic and hydrophilic phases to form a two-phase mixture, and

d) adding an emulsifier to the two-phase mixture to form an emulsion.

Furthermore, they may contain other common ingredients of creams and may be formulated in a manner that is well known to those skilled in the art.

Advantageously, the medicaments according to the present invention contains at least one inactive ingredient selected from among butylated hydroxytoluene, cetyl alcohol, citric acid, glycerol, glyceryl stearate, magnesium aluminum silicate, methyl gluceth-10, methyl paraben, PEG-100 stearate, propyl paraben, purified water, sodium metabisulfite, stearic acid and stearyl alcohol.

Advantageously, the medicaments according to the present invention include the cream Tri-Luma® marketed by Galderma, as presented in Example 1.

In order to further illustrate the present invention and the advantages thereof, the following specific examples are given, it being understood that same are intended only as illustrative and in nowise limitative. In said examples to follow, all parts and percentages are given by weight, unless otherwise indicated.

### Example 1

### Composition of the Cream Tri-Luma®

The cream contains, in weight percentage relative to the total weight:

| | |
|---|---|
| magnesium aluminum silicate | 3.00% |
| butylated hydroxytoluene | 0.04% |
| cetyl alcohol | 4.00% |
| stearic acid | 3.00% |
| stearyl alcohol | 4.00% |
| methyl paraben | 0.18% |
| propyl paraben | 0.02% |
| Arlacel ® 165 (glyceryl stearate and | 3.50% |
| glyceryl monostearate PEG-100 stearate] | |
| methyl gluceth-10 | 5.00% |
| glycerol | 4.00% |
| tretinoin | 0.05% |
| fluocinolone acetonide | 0.01% |
| citric acid | 0.05% |
| hydroquinone | 4.00% |
| sodium metabisulfite | 0.20% |
| purified water | 68.95% |

### Example 2

### Study of the Efficacy of Tri-Luma® on Various Signs of Photoaging of the Skin

60 individuals from 40 to 82 years old were randomized into two groups of 30 people and were treated on the face, respectively, with the product Tri Luma and the second group with 0.025% tretinoin alone (Vitanol A) for 8 weeks of treatment.

Various criteria were measured via a score of severity (from Absent to Severe or Very Severe) at time T0 before treatment and at the end of the treatment: score for mottled hyperpigmentation, for the severity of the photoaging and for the severity of the fine lines.

The improvement or worsening relative to the initial state before treatment (T0) was measured for each individual by means of the After-Before treatment difference (T8-T0). A negative difference in score thus means an improvement (and vice-versa for a positive difference).

The table below presents, for each measured criterion, the distribution of the individuals in each category of score difference, and also the analysis of the comparison from the treatments.

| | | Tri-Luma | Vitanol A | p-Value |
|---|---|---|---|---|
| Facial mottled hyperpigmentation | | | | |
| Visit at T8 | Number of | 100.0% | 100.0% | |
| weeks | individuals = 30 | | | |
| | 0 | 36.7% | 43.3% | |
| | −1 | 33.3% | 50.0% | |
| | −2 | 30.0% | 6.7% | |
| | Lsmean ± Std err | −0.9 ± 0.1 | −0.6 ± 0.1 | 0.091 |
| | Mean ± sd | −0.9 ± 0.8 | −0.6 ± 0.6 | |
| Measurement of the severity of | | | | |
| photoaging | | | | |
| Visit at T8 | Number of | 100.0% | 100.0% | |
| weeks | individuals = 30 | | | |
| | 0 | 60.0% | 60.0% | |
| | −1 | 23.3% | 36.7% | |
| | −2 | 16.7% | 3.3% | |
| | Lsmean ± Std err | −0.6 ± 0.1 | −0.4 ± 0.1 | 0.502 |
| | Mean ± sd | −0.6 ± 0.8 | −0.4 ± 0.6 | |
| Measurement of the severity of | | | | |
| the fine lines | | | | |
| Visit at T8 | Number of | 100.0% | 100.0% | |
| weeks | individuals = 30 | | | |
| | 1 | 3.3% | 3.3% | |
| | 0 | 66.7% | 70.0% | |
| | −1 | 26.7% | 26.7% | |
| | −2 | 3.3% | — | |
| | Lsmean ± Std err | −0.3 ± 0.1 | −0.3 ± 0.1 | 0.968 |
| | Mean ± sd | −0.3 ± 0.6 | −0.2 ± 0.5 | |

These results show that the product Tri-Luma has activity on the signs associated with photoaging and more particularly on mottled hyperpigmentation and wrinkles. This product is much more effective in the treatment of the signs of photoaging of the skin than tretinoin alone.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for treating the signs of photoaging of the skin, comprising administering to a subject in need of such treatment, a thus effective amount of a pharmaceutical/dermatological medicament which comprises a combination of fluocinolone acetonide, hydroquinone and tretinoin.

2. A regime or regiment for treating cutaneous hyperpigmentation marks induced by exposure to ultraviolet radiation, comprising administering to a subject in need of such treatment, a thus effective amount of a pharmaceutical/dermatological medicament which comprises a combination of fluocinolone acetonide, hydroquinone and tretinoin.

3. A regime or regimen as defined by claim 1 for treating the signs of photoaging of the skin selected from the group consisting of actinic keratosis, mottled hyperpigmentation, solar lentigo, flat pigmented seborrhoeic warts, wrinkles and fine lines, solar elastosis, skin roughness, and freckles, comprising administering to a subject in need of such treatment, a thus effective amount of a pharmaceutical/dermatological medicament which comprises a combination of fluocinolone acetonide, hydroquinone and tretinoin.

4. The regime or regimen as defined by claim 1, wherein the signs of photoaging of the skin are selected from among actinic keratosis, mottled hyperpigmentation, solar lentigo and flat pigmented seborrhoeic warts.

5. The regime or regimen as defined by claim 1, said pharmaceutical/dermatological medicament being topically applied onto the affected skin area of said subject.

6. The regime or regimen as defined by claim 1, said pharmaceutical/dermatological medicament comprising from 1% to 10% by weight of hydroquinone relative to the total weight thereof.

7. The regime or regimen as defined by claim 1, said pharmaceutical/dermatological medicament comprising from 2% to 7% by weight of hydroquinone relative to the total weight thereof.

8. The regime or regimen as defined by claim 1, said pharmaceutical/dermatological medicament comprising from about 4% by weight of hydroquinone relative to the total weight thereof.

9. The regime or regimen as defined by claim 1, said pharmaceutical/dermatological medicament comprising from 0.025% to 2% by weight of tretinoin relative to the total weight thereof.

10. The regime or regimen as defined by claim 1, said pharmaceutical/dermatological medicament comprising from 0.025% to 1% by weight of tretinoin relative to the total weight thereof.

11. The regime or regimen as defined by claim 1, said pharmaceutical/dermatological medicament comprising from about 0.05% by weight of tretinoin relative to the total weight thereof.

12. The regime or regimen as defined by claim 1, said pharmaceutical/dermatological medicament comprising from 0.005% to 0.1% of fluocinolone acetonide by weight relative to the total weight.

13. The regime or regimen as defined by claim 1, said pharmaceutical/dermatological medicament comprising from 0.005% to 0.05% of fluocinolone acetonide by weight relative to the total weight.

14. The regime or regimen as defined by claim 1, said pharmaceutical/dermatological medicament comprising from about to 0.01% of fluocinolone acetonide by weight relative to the total weight.

15. The regime or regimen as defined by claim 1, said pharmaceutical/dermatological medicament being formulated as a cream.

16. The regime or regimen as defined by claim 1, said pharmaceutical/dermatological medicament comprising, in weight percentage relative to the total weight:

| | |
|---|---|
| magnesium aluminum silicate | 3.00% |
| butylated hydroxytoluene | 0.04% |
| cetyl alcohol | 4.00% |
| stearic acid | 3.00% |
| stearyl alcohol | 4.00% |
| methyl paraben | 0.18% |
| propyl paraben | 0.02% |
| Arlacel ® 165 (glyceryl stearate and | 3.50% |
| glyceryl monostearate PEG-100 stearate] | |
| methyl gluceth-10 | 5.00% |
| glycerol | 4.00% |
| tretinoin | 0.05% |
| fluocinolone acetonide | 0.01% |
| citric acid | 0.05% |
| hydroquinone | 4.00% |
| sodium metabisulfite | 0.20% |
| purified water | 68.95% |

17. The regime or regimen as defined by claim 1, said pharmaceutical/dermatological medicament further comprising a hydroquinone anti-oxidizing amount of sodium metabisulfite.

18. A pharmaceutical/dermatological medicament useful for treating the signs of photoaging of the skin, comprising a combination of fluocinolone acetonide, hydroquinone and tretinoin, formulated into a physiologically acceptable medium therefor.

19. The pharmaceutical/dermatological medicament as defined by claim 18, formulated for topical application.

20. The pharmaceutical/dermatological medicament as defined by claim 19, formulated as a cream.

21. The pharmaceutical/dermatological medicament as defined by claim 18, further comprising a hydroquinone anti-oxidizing amount of sodium metabisulfite.

22. The pharmaceutical/dermatological medicament as defined by claim 18, comprising from 1% to 10% by weight of hydroquinone.

23. The pharmaceutical/dermatological medicament as defined by claim 22, comprising from 0.025% to 2% by weight of tretinoin.

24. The pharmaceutical/dermatological medicament as defined by claim 23, comprising from 0.005% to 0.1% by weight of fluocinolone acetonide.

25. The pharmaceutical/dermatological medicament as defined by claim 18, comprising in weight percentage relative to the total weight thereof:

| | |
|---|---|
| magnesium aluminum silicate | 3.00% |
| butylated hydroxytoluene | 0.04% |
| cetyl alcohol | 4.00% |
| stearic acid | 3.00% |
| stearyl alcohol | 4.00% |
| methyl paraben | 0.18% |
| propyl paraben | 0.02% |
| Arlacel ® 165 (glyceryl stearate and | 3.50% |
| glyceryl monostearate PEG-100 stearate] | |
| methyl gluceth-10 | 5.00% |
| glycerol | 4.00% |
| tretinoin | 0.05% |
| fluocinolone acetonide | 0.01% |
| citric acid | 0.05% |
| hydroquinone | 4.00% |
| sodium metabisulfite | 0.20% |
| purified water | 68.95% |
